(19) United States
(12) Patent Application Publication
(10) Pub. No.: US 2015/0105697 A1
(43) Pub. Date: Apr. 16, 2015

(54) APPARATUS AND METHOD FOR DETECTING DIASTASIS RECTI

(71) Applicant: TUPLER INC., New York, NY (US)

(72) Inventor: Julie Tupler, New York, NY (US)

(21) Appl. No.: 14/453,214

(22) Filed: Aug. 6, 2014

Related U.S. Application Data
(60) Provisional application No. 61/891,606, filed on Oct. 16, 2013.

(51) Int. Cl. A61B 5/107

(52) U.S. Cl. CPC A61B 5/1072
USPC 600/587

(57) ABSTRACT

A measuring kit for determining parameters indicative of a DR (diastasis recti) condition of a patient includes two components: a distance measuring component adapted to measure a distance DR parameter and a depth measuring component adapted to measure a depth DR parameter of a user. The measurements obtained with these components are compared with standard measurements obtained from other users and used to indicate or determine the condition of the user. The kit is particularly helpful in monitoring the progress (or lack of progress) of a person performing certain exercises designed to alleviate DR conditions.

### RELATED APPLICATIONS

This application claims priority to provisional application Ser. No. 61/891,606 filed Oct. 16, 2013 and incorporated herein in its entirety.

### BACKGROUND OF THE INVENTION

A. Field of Invention

This invention pertains to a method and apparatus for detecting and quantifying the diastasis recti (or DR) of a person. The apparatus includes a tape preferably secured around the waist of a person with indicia provided on the tape for determining and indicating the lateral separation associated with the person. The apparatus further includes a finger component that is also provided with indicia. The finger component is donned over a finger and used to determine a depth related to DR.

B. Description of the Prior Art

Diastasis recti (hereinafter “DR”) or abdominal separation refers to a separation of the abdominal muscles of a person. Although generally an abnormally large DR is associated with infants or women (especially after childbirth), the present inventor believes that this condition is much more prevalent in all people, and that, if left untreated, can result in various medical problems, some of which could be disabling, including hernias, lower back pain and other complications.

Of course before one treats a condition such as DR, it is important that a person or a care provider determine quantitatively whether DR is present and its severity. However, until now there has been no apparatus or device in place for measuring quantitatively characteristics or parameters associated with DR. A diastasis is presently checked with the number of fingers that can fit in between the separated muscles.

### SUMMARY OF THE INVENTION

The present invention provides a tape measure belt and finger sock which can successfully be used by one person to measure the width and depth of the DR of a person. Once the apparatus is used, measurements obtained from a plurality of patients and other users are used to standardize such measurements and derive several ranges for these parameters that provide some indication of the DR condition of a patient.

The apparatus and method can therefore be used by people who wish to determine the size of their diastasis by themselves, and can but need not rely upon an assistant. Furthermore, the invention sets a standard system of measurement in place for the determination of the size and depth of a diastasis recti, whereas none has previously been universally utilized.

In one embodiment of the invention, a measuring tape with two sliding elements disposed at each end to elastic strips backed with Velcro® or other similar attachment or fastening means. This measuring device is worn as a belt encircling the user's torso. A reference point such as a red dot is positioned at the center of the measuring strip in alignment with the wearer's navel.

The measuring device is readily operated by one person, and the values are obtained by sliding the indicator loops into position, thereby marking the location of the user's diastasis recti and reading one of its characteristic parameters from the location of the indication loops with respect to the indicia on the tape.

A second element consists of a finger sock with measurement markings which, can easily be used by one person to determine the depth of the diastasis recti by gently depressing the finger-sock-clad forefinger into the abdomen at the naval and at 3 inches above and below the naval. The lowest measurement visible is the depth of the diastasis at that point on the abdomen.

One aspect of the invention is that for the first time it provides an anatomical measuring device, which is readily operable by one person without the need of an assistant, allowing the user to determine the width and depth of one's diastasis recti.

Another aspect of the invention is that it sets in place a standardized measuring system to accurately describe the width and depth of a diastasis recti of a person, whereas no such standardized unit of measure has previously been in place.

A somewhat general aspect of the invention to provide an apparatus for the purposes described above which is inexpensive, easy to use, effective I and creates a universal system of measuring a diastasis recti, either by a person on him- or her-self or by a health care provider. Once it is determined that a person has an abnormal DR, he or she is prescribed a series of exercises. The apparatus and method is then used to accurately monitor the DR condition and determine if there is any progress closing (or re-opening) of his/her diastasis recti and thereby tailor his/her fitness regimen and activities accordingly.

### BRIEF DESCRIPTION OF THE DRAWINGS

Various other objects, features, and attendant advantages of the present invention will become more fully appreciated as the same becomes better understood when considered in conjunction with the accompanying drawings, wherein:

FIG. 1A shows a plan view of a measuring belt constructed in accordance with this invention;

FIG. 1B shows a plan view of a tape forming a central portion of the measuring belt of FIG. 1A and having DR indicia;

FIG. 1 is an environmental top view of a user lying down on the floor, wearing the measuring belt element of the invention, showing the first measurement step of feeling for the ridges of the diastasis with the fingers and adjusting the indicator loop to mark the outer location of the diastasis, preferably performed with the head raised off the floor;

FIG. 2 is a close-up top view of the user lying on the floor, wearing the measuring belt element of the invention, showing the second measurement step of feeling for the opposite-side ridge of the diastasis with the fingers and adjusting the other indicator loop to mark the opposite side of the diastasis;

FIG. 3 is an environmental top view of user lying down on the floor, wearing the measuring belt element of the invention, showing how the user must use two hands to feel for the diastasis in cases where the diastasis creates an outwardly protrusion on the abdomen, preferably with one hand being removed to slide the first loop to the position of the finger of the other hand marking the ridge; after the loop is in place, the user must then alternate hands and again feel for the opposite-side ridge and slide the other loop to its location;

FIG. 4 is an environmental frontal view of user lying down on the floor, wearing the sock on the index finger and gently depressing it into the navel; and

FIG. 5 shows the sock worn on the forefinger; and

FIG. 5A shows an enlarged perspective view of the sock.

### DETAILED DESCRIPTION OF THE INVENTION

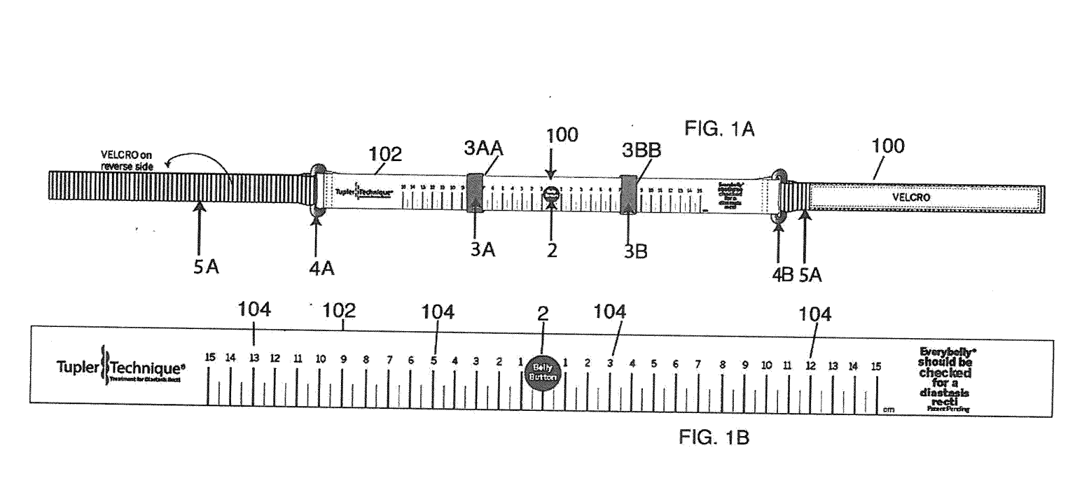

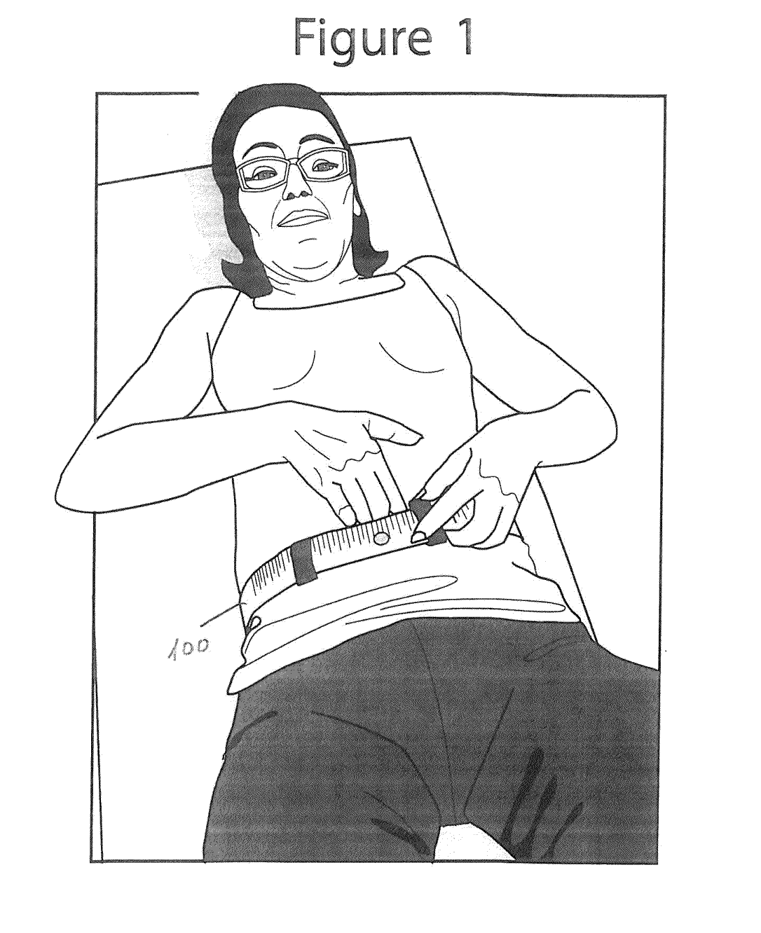

Referring first to FIGS. 1A and 1B, a measuring device 100 includes a tape 102. The tape 102 is attached by metal rings 4A, 4B to elastic bands 5A, 5B. The measuring device 100 is placed around the waist of a person (as discussed in more detail below) with a central dot 2 positioned over the naval. The elastic bands 5, wrap around the user's torso and fasten in the back with the attached Velcro strips 104A, 104B. Tape 102 has indicia that can be used to quantify the positions of the edges of the abdominal muscles of a person, for example with respect to the navel.

In one embodiment, the indicia 104 is presented in the form of vertical lines or gradations preferably starting from the central dot 2 toward the edges of the tape terminating with rings 4A, 4B. The gradations 104 are in centimeters, inches, or any other convenient units.

Ends 5A, 5B attached to tape 102 are used to selectively mount device 100 around the torso of a user and are arranged and constructed so that they can be secured to each other so that the device 100 does not fall off or otherwise slip away while measurements are taken. Preferably the apparatus 100 is disposed along a plane perpendicular to the spine of the user. The ends 5A, 5B can be secured to each other by well known means as tying, Velcro® type hook and loop elements attached respectively to ends 5A, 5B., snaps, zippers, releasable adhesives, etc.

The device 100 is securely fastened around the torso as the user lies down on the floor with knees bent and the head lifted slightly off the ground as shown in FIG. 1. Placing the hand on one side of the naval he or she must feel with the fingers the location of the of the ridge of the abdominal muscles (not shown). Then the other side of the naval needs to be checked. The positions of these ridges on each side are indicative of the abdominal muscle separation. If a user is unable to locate this ridge (not shown) he or she should bring the head up and down several times until they feel the movement of the muscles coming together.

Once the user locates a ridge (for example, the ridge on the left side in FIG. 1) with the right hand, the user positions an indicator loop (in this case loop 3B, with the other hand, by sliding the loop 3B along the tape 102 so that its inner edge 3BB (nearest the navel) marks the position of the left abdominal muscle (and therefore one edge of the diastasis).

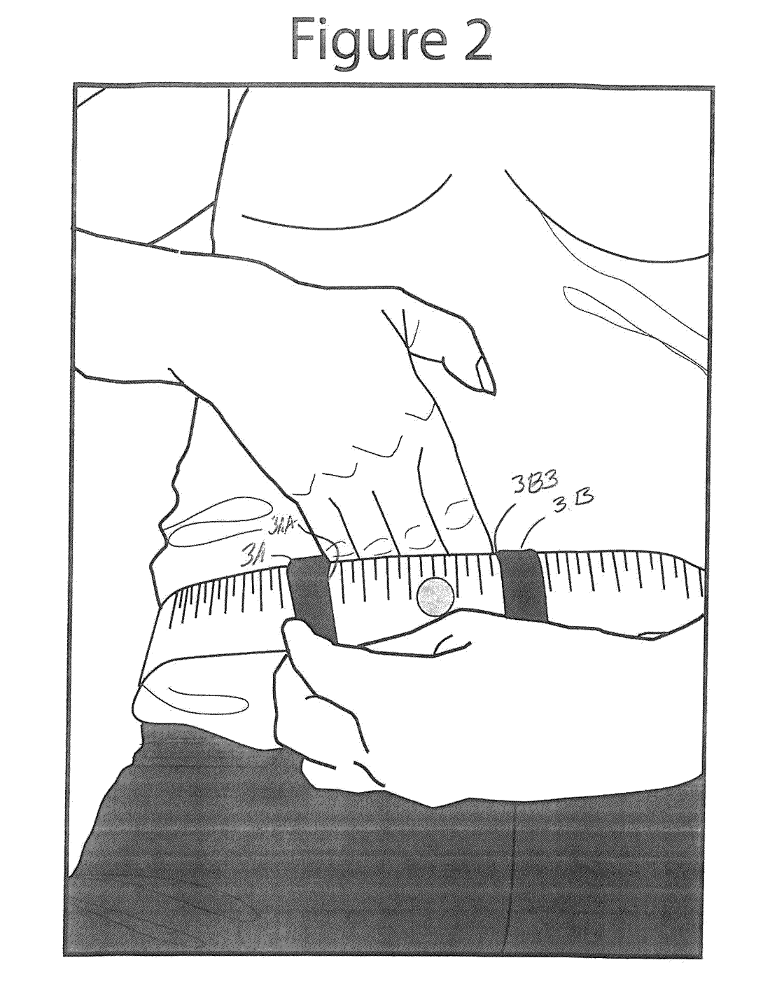

Then the user locates the ridge on the opposite (right) side of the naval (see FIG. 2) and slides the other indicator loop 3A with his left hand so that its edge 3AA marks this right ridge.

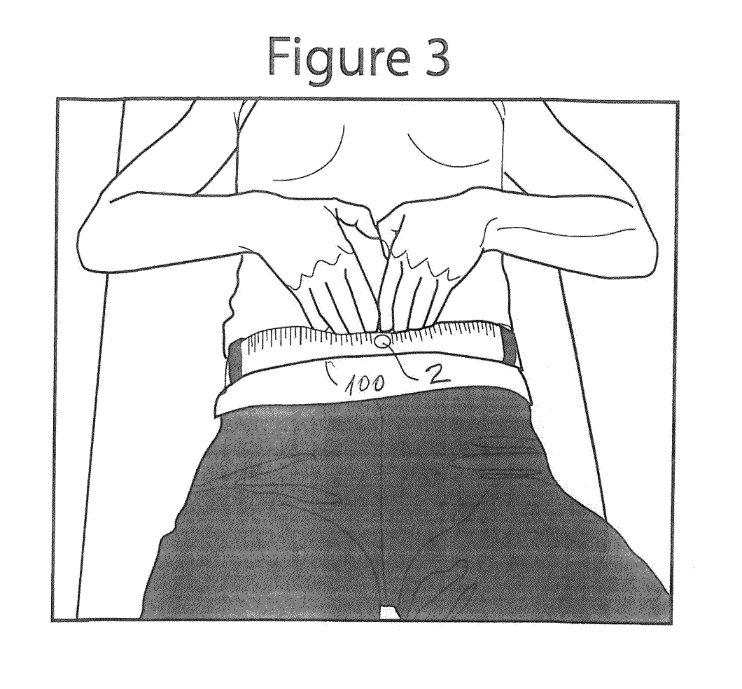

If a user has a protruding diastasis, he may use both hands to locate the ridges (see FIG. 3) on each side and then he can withdraw one hand at a time to slide and position the indicator loops 3A, 3B as discussed above.

When both sides of the diastasis (both ridges) have been located and designated by moving the indicator loops into the correct position, the user carefully sits up, making sure not to move the loops 3A, 3B, and he/she looks down at the tape 102 to read the positions of the indicator loops 3A, 3B as provided by the gradations 104.

In one embodiment, the two respective positions of the loops provide the right and left lateral ridge positions of the abdominal muscles can be used as DR parameters. In another embodiment, the total distance between the ridges is used as a DR parameter and this DR parameter is obtained by adding the two gradations of the two loops together. This sum is the width (in centimeters or other appropriate units) of the diastasis recti at the naval.

The steps above are now repeated, first moving the belt 100 3 inches above the navel. The central dot 2 again aligned with (but above) the naval. Again the user lies down on the floor with knees bent and lifts the head slightly off the ground. With the fingers placed at the top edge of the belt approximately 3 inches above the navel, he or she repeats the process of feeling for the ridges of the diastasis and sliding the indicator loops 3A, 3B so that their respective inner edges 3AA, 3BB is disposed at the correct locations. The same process is then repeated 3 inches below the navel. The distance in centimeters, equaling the width of the diastasis, must be determined at both locations, 3 inches above and 3 inches below the navel.

Thus, as described above, the device 100 is used to generate anywhere from one to six distance parameters descriptive of a DR condition: the distance between the loop edges 3AA-3BB taken at a preselected position, e.g., with the central dot 2 overlaying the naval of the user; the three distances measured at three levels—at the naval, 3 inches above the navel and 3 inches below the navel; the six distances between the reference central dot 2 and edges 3AA, 3BB taken at the three levels, etc.

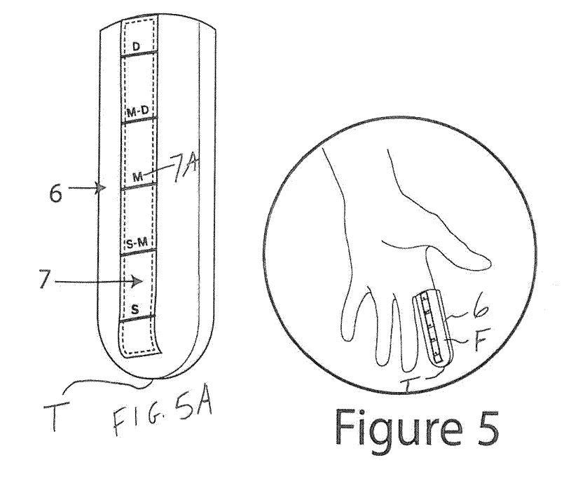

According to the invention, a second device is also provided to measure yet another parameter related to DR, such as a depth parameter. This measures the condition of the connective tissue. The further down the finger goes the weaker the connective tissue. Referring first to FIG. 5A, a device for measuring the depth parameter may be implemented as a finger sock 6 that is preferably worn on the forefinger of the user. The sock 6 may be made of any known techniques, including weaving, knitting, etc, and can be made from any artificial or natural yams, or blends thereof. The sock 6 should be somewhat flexible and extendable so that it can fit tightly on the finger F of a person, whether that person has a large, medium or small forefinger.

Attached to an outer surface of the sock 6 and extending generally longitudinally is a tape 7, Tape 7 is preferably made of a material that does not shrink or expand at least longitudinally. A plurality of indicia 7A are provided on tape 7. Again the indicia may be gradations indicative of a distance from the tip T of the finger as measured in centimeters, inches, etc.

Alternatively, the indicia may be letters, as indicated in FIG. 5A representing the depth parameter for a DR as described below. In one embodiment, these letters may include;

S=shallow

S-M=Shallow/medium

M=medium

M-D=Medium/Deep

D=Deep

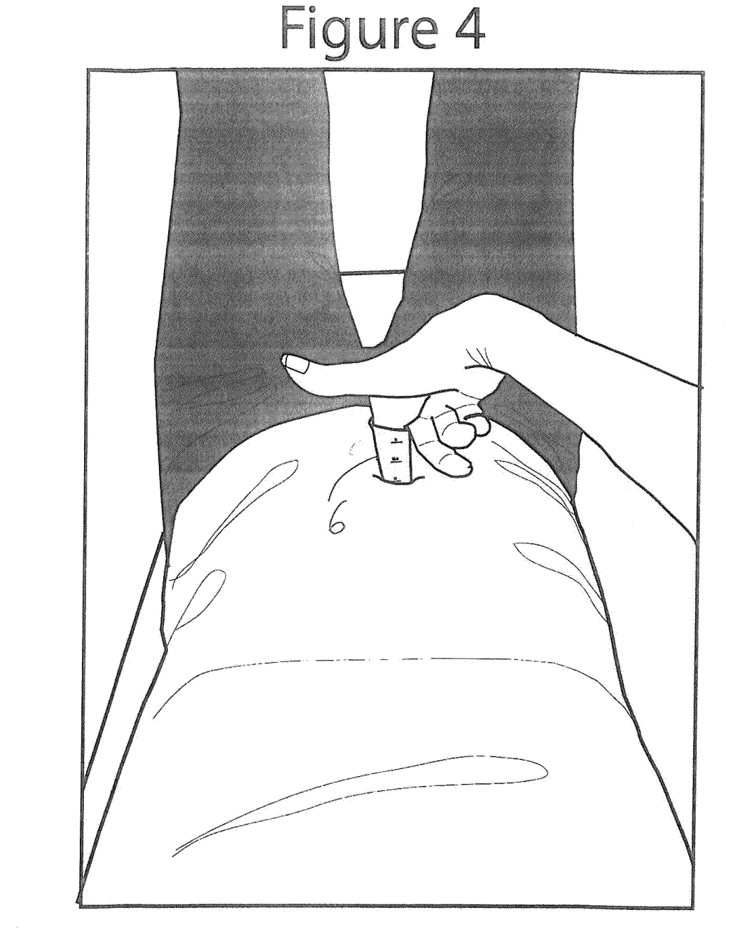

Device 6 is used as follows. A person or user slips the finger sock 6 over the a finger F (such as the index or forefinger F in FIG. 5), lies down and insert the respect finger in his/her belly button as shown in FIG. 4. The forefinger F with the tape 7 facing upward so that it is visible to the user. Without forcing the connective tissue down, the user should feel for the bottom of the gulley, or bottom of the diastasis. The marking is still visible on the bottom-most part of the sock 6 is used as an indication (e.g. depth parameter) indicates the depth of the diastasis at the naval, e.g., S, S-M, M, etc.

Again, a single depth parameter may be obtained, for example at the naval, or several depth parameters may be obtained at different levels in the same locations as when measuring the distance.

In the discussion provided above, the measurements for obtaining the distance and depth parameters are performed by the user—e.g a person with a normal or potentially abnormal DR. Of course, instead of the user, a health care provider may obtain these measurements, especially when the user is not very familiar with DR and/or the apparatus and method described herein.

The measuring device 100 is preferably packaged with a sock 6 and sold to the public as a kit together, with instructions to use them to determine DR related distance and depth parameters.

The order in which the parameters, e.g. the distance DR parameters from loops 3A, 3B and the depth DR parameters obtained from sock 6 does not matter. In other words, the distance parameters can be obtained first, followed by the depth DR parameter(s) and so on.

Measurements obtained from a preselected population may be averaged or otherwise processed using known techniques such as statistical analysis. The results are then used as a standard to determine the actual condition of a patient, For example, the following are some standardized readings that have been obtained or deduced by the inventor:

| PARAMETER | NORMAL | MILD DR | MEDIUM DR | SEVERE DR |
|---|---|---|---|---|
| Distance | 2 cm | 4 cm | 8 cm | 14 cm |
| Depth | S | SM | M | MD |

In the above table, the distance parameter was measured at the naval level with edges being equidistant from center point 2.

Numerous modifications may be made to this invention, without departing from its scope as defined in the appended claims.

1. A kit for measuring parameters related to the DR (diastasis recti) condition of a user, said kit comprising:
a distance measuring device including a tape portion formed of an elongated tape having a plurality of gradations disposed longitudinally along said tape, fastening elements attached to the ends of the tape portion, the fastening elements cooperating to hold said distance measuring devices mounted around the torso of the user; and at least a first ridge position selecting element slidably mounted on said elongated tape and adapted to be positioned at a location corresponding the position of a ridge of the abdominal muscle of a person, the distance between said location and a reference point on the tape defining a distance DR parameter descriptive of the DR condition of the user; and
a depth measuring device including a sock sized and shaped to fit on the finger of a user, said sock including an outer surface with a longitudinal element having depth gradations, wherein when said depth measuring device is pushed inward in the abdominal area of the user, the depth gradations are indicative of the depth DR parameter of the user.

2. The kit of claim 1 wherein said tape includes a central point and said first ridge position selecting element has a location indicative of the distance between the naval of the person and the ridge.

3. The kit of claim 1 further comprising a second ridge position element slidable on said tape, wherein when said distance measuring device is positioned laterally around the torso of the user, the first and second ridge position element indicate distances of the respective ridges of the abdominal muscle of the user.

4. The kit of claim 3 wherein said tape includes a central portion and said first and second ridge position elements are disposed on either side of said central portion element, said central portion element being placed at the naval of the user to provide said distance DR parameter.

5. A method of generating a plurality of DR indicative parameters indicative of the DR condition of a patient comprising the steps of
placing a distance measuring device around a patient, the distance measuring device including a central tape with gradations and a first ridge position selecting element movable with respect to said tape;
detecting at least one ridge of the abdominal muscle of the patient;
positioning the first ridge position selecting element at the one ridge, the location of the first ridge selecting element with respect to the tape indicating at least one distance DR parameter;
mounting a sock on the finger of the user, the finger, said finger including a longitudinal set of gradations, and
pushing the finger with the sock into the user body near the navel area to determine a depth DR parameter, said depth DR parameter being determined from the visible gradations.

6. The method of claim 5 wherein said distance measuring device includes a second ridge position element movable along the tape, comprising the step of moving the second ridge position element to the second ridge of the abdominal muscle, the location of the second ridge position element, the distance DR parameter being dependent on the location of the first and the second ridge position elements.
